(19) United States
(12) Patent Application Publication
(10) Pub. No.: US 2007/0014869 A1
(43) Pub. Date: Jan. 18, 2007

(54) Compositions for reconstruction, replacement or repair of intracardiac tissue

(75) Inventor: Robert G. Matheny, Norcross, GA (US)

Correspondence Address: LEIGH HUNT FIRESTONE, 7100 NORFOLK ROAD, BERKELEY, CA, 94705, US

(73) Assignee: CorMatrix Cardiovascular, Inc., Santa Cruz, CA (US)

(21) Appl. No.: 11/334,631

(22) Filed: Jan. 18, 2006

Related U.S. Application Data
(63) Continuation-in-part of application No. 11/182,551, filed on Jul. 15, 2005.

(51) Int. Cl. A61K 35/38; A61K 35/37

(52) U.S. Cl. 424/551

(57) ABSTRACT

The invention is to a composition for reconstruction, replacement or repair of a defect in or dsmage to intracardiac tissue comprising a patch, emulsion, injectable solution or other composition comprising extracellular matrix. As extracellular matrix can assimilate with the intracardiac tissue to help generate healthy functioning tissue, using extracellular matrix to address a defect in intracardiac tissue can obviate the need for second or subsequent open heart surgeries, or if the composition can be delivered in a minimally invasive medical procedure, can obviate the need for open heart surgery altogether. Conduction of the intracardiac tissue may also be restored using extracellular matrix material to reconstruct, replace or repair the damaged intracardiac tissue.

### CROSS-REFERENCES TO RELATED APPLICATIONS

The present application is a continuation-in-part of U.S. application Ser. No. 11/182,551, filed Jul. 15, 2005, to which priority is claimed.

### FIELD OF THE INVENTION

The invention relates to tissue reconstruction, replacement or repair generally, and more specifically to compositions and methods for reconstruction, replacement or repair of damaged or defective intracardiac tissue.

### BACKGROUND OF THE INVENTION

Each year in the United States alone over 600,000 open heart surgeries are conducted, and about 20% of these are on children and adults to address one or more defects in intracardiac tissue. Where a prosthetic device or material is used in these surgeries to correct the defect, many times the surgery will need to be repeated as the child grows, replacing the prosthetic item with a larger version to accommodate the growing organ in the growing child. Likewise, intracardiac defects in adults presently require open heart surgery, at a coordinate mortality risk due to the nature of the surgery alone. Repeat operations are common with heart valve replacements when the prosthetic valve needs to be replaced.

Accordingly, it would be a great advantage to the medical community for repair of damage or defects in intracardiac tissue, to provide an invention that either eliminates the need for open heart surgery in order to reconstruct, replace or repair the damaged or defective intracardiac tissue, or to provide a material that will become the patient's own tissue and grow and assimilate with the intracardiac tissue and obviate the need for a second or subsequent open heart surgeries. Presently, no such product or method exists on the market to address the challenge of reconstructing, replacing or repairing defects in intracardiac tissue.

A function of the intracardiac tissue is electrical conduction of the heart. When the sino atrial or atrio ventricular nodes are not functioning properly, the electrical connections required for proper heart function are not being generated. The sino atrial node (often called the SA node or sinus node) serves as the natural pacemaker for the heart. Nestled in the upper area of the right atrium, it sends electrical impulse that triggers each heart beat. The impulse spreads through the atria, prompting the cardiac muscle tissue to contract in a coordinated wave-like manner. The impulse that originates from the sino atrial node strikes the atrio ventricular node (or AV node) which is situated in the lower portion of the right atrium. The atrio ventricular-node in turn sends an impulse through the nerve network to the ventricles, initiated the same wave-like contraction of the ventricles. The electrical network serving the ventricles leaves the atrio ventricular node through the right and left bundle branches. These nerve fibers send impulses that cause the cardiac muscle tissue to contract. When the conduction pathway between the sino atrial and atrio ventricular nodes fails, or when either the sino atrial or atrio ventricular node repair is necessary, patients are implanted with pacemaker leads to re-establish the proper conduction impulses that cause the intracardiac muscle tissue to contract, and which may has been partially lost due to damage or defect to the intracardiac tissue in these regions. The damage can be due to aging. The patient with pace maker leads controlled by an external pacemaker is forever dependent on an external mechanical device (the pacemaker) to generate proper electrical conduction in the heart.

It would be a great advantage to the medical community generally and cardiac patients specifically to provide new ways to repair damaged or defective intracardiac tissue, including the damage that can occur with aging or trauma to the conduction pathways in the heart.

### SUMMARY OF THE INVENTION

The invention provides a composition for reconstruction, replacement or repair of damaged or defective intracardiac tissue comprising extracellular matrix, wherein the composition comprises a form selected from the group consisting of a patch, an emulsion, an injectable solution, a gel, a fluid, a paste, a powder, a strand, a strip, a spray, a vapor, a cream, and a coating. The composition can comprise mammalian or synthetic extracellular matrix.

The invention is also a composition for reconstruction, replacement or repair of damaged or defective intracardiac tissue comprising extracellular matrix, where said composition comprises a form selected from the group consisting of a patch, an emulsion, an injectable solution, a gel, a fluid, a paste, a powder, a strand, a strip, a spray, a vapor, a cream, and a coating, and where said composition further comprises an additional component selected from the group of:

a) a cell,

b) a peptide, polypeptide, or protein,

c) a vector having a DNA capable of targeted expression of a selected gene, and

d) a nutrient, a sugar, a fat, a lipid, an amino acid, a nucleic acid, a ribo-nucleic acid, an organic molecule, an inorganic molecule, a small molecule, a drug, or a bioactive molecule.

The damage or defect in intracardiac tissue can comprise damage or defect to a sino atrial node, an atrio ventricular node, or to a conduction pathway in between the two.

Further to the invention is provided a method of reconstruction, replacement or repair of damaged or defective intracardiac tissue, comprising contacting the damage or defect with a composition for reconstruction, replacement or repair of an intracardiac defect comprising extracellular matrix, wherein the composition comprises a form selected from the group consisting of a patch, an emulsion, an injectable solution, a gel, a fluid, a paste, a powder, a strand, a strip, a spray, a vapor, a cream, and a coating.

Also the invention provides a method of reconstruction, replacement or repair of damaged or defective intracardiac tissue, comprising contacting a damaged or defective site with a composition for reconstruction, replacement or repair of damaged or defective intracardiac tissue comprising extracellular matrix, where said composition comprises a form selected from the group consisting of a patch, an emulsion, an injectable solution, a gel, a fluid, a paste, a powder, a strand, a strip, a spray, a vapor, a cream, and a coating, and where said composition further comprises an additional component selected from the group of:

a) a cell,

b) a peptide, polypeptide, or protein,

c) a vector having a DNA capable of targeted expression of a selected gene, and

d) a nutrient, a sugar, a fat, a lipid, an amino acid, a nucleic acid, a ribo-nucleic acid, an organic molecule, an inorganic molecule, a small molecule, a drug, or a bioactive molecule.

The methods can be practiced wherein contacting comprises surgically opening the patient at a site of damage or defect and placing the composition at the site. Alternatively, contacting can comprise delivering the composition to a site of damage or defect in the intracardiac tissue using a catheter in a minimally invasive procedure.

Also provided is a method of making a composition for reconstruction, replacement or repair of damaged or defective intracardiac tissue comprising extracellular matrix, wherein the composition comprises a form selected from the group consisting of a patch, an emulsion, an injectable solution, a gel, a fluid, a paste, a powder, a strand, a strip, a spray, a vapor, a cream, and a coating, said method comprising preparing the composition, and packaging the composition for storage until use.

Further is provided a method of making a composition for reconstruction, replacement or repair of damaged or defective intracardiac tissue comprising extracellular matrix, where said composition comprises a form selected from the group consisting of a patch, an emulsion, an injectable solution, a gel, a fluid, a paste, a powder, a strand, a strip, a spray, a vapor, a cream, and a coating, and where said composition further comprises an additional component selected from the group of:

a) a cell,

b) a peptide, polypeptide, or protein,

c) a vector having a DNA capable of targeted expression of a selected gene, and

d) a nutrient, a sugar, a fat, a lipid, an amino acid, a nucleic acid, a ribo-nucleic acid, an organic molecule, an inorganic molecule, a small molecule, a drug, or a bioactive molecule, said method comprising preparing the composition, and packaging the composition for storage until use.

The damaged or defective intracardiac tissue can comprise intracardiac tissue in a sino atrial node, an atrio ventricular node, or a conduction pathway in between the two.

### BRIEF DESCRIPTION OF THE DRAWINGS

FIG. 1 depicts cell-extracellular matrix interaction through the matrix proteoglycans, glycoaminoglycans and growth factors.

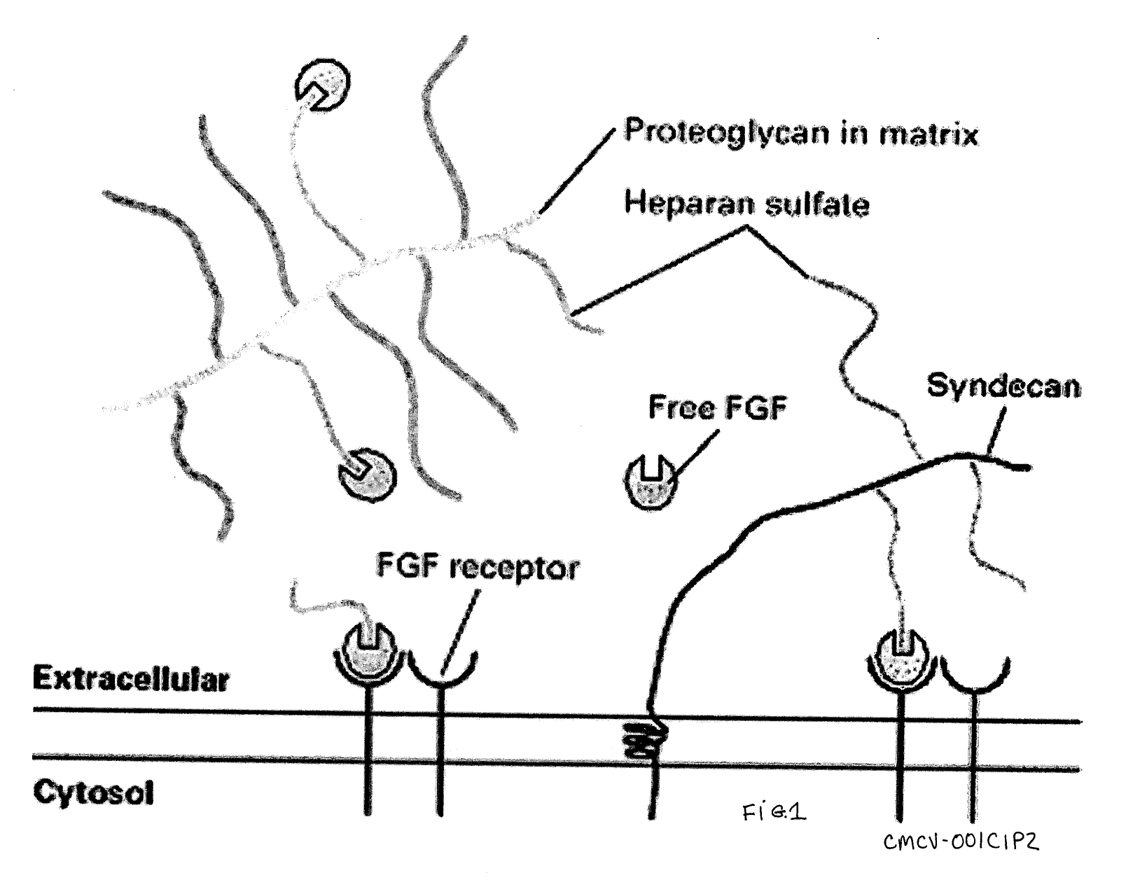

FIG. 2 depicts cell-cell adhesions, and cell-matrix adhesions through specific structural and functional molecules of the extracellular matrix.

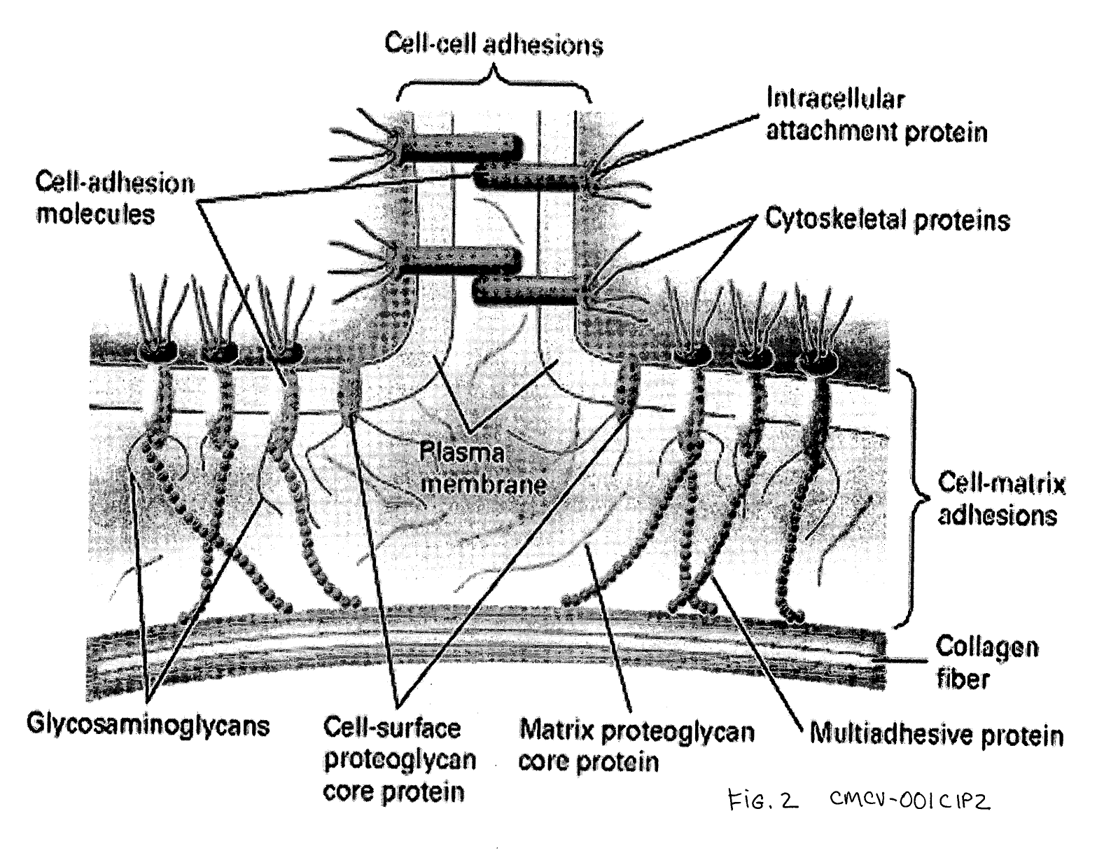

FIG. 3 depicts a model of matrix scaffold structure including common collagen, proteoglycans, and glycoproteins.

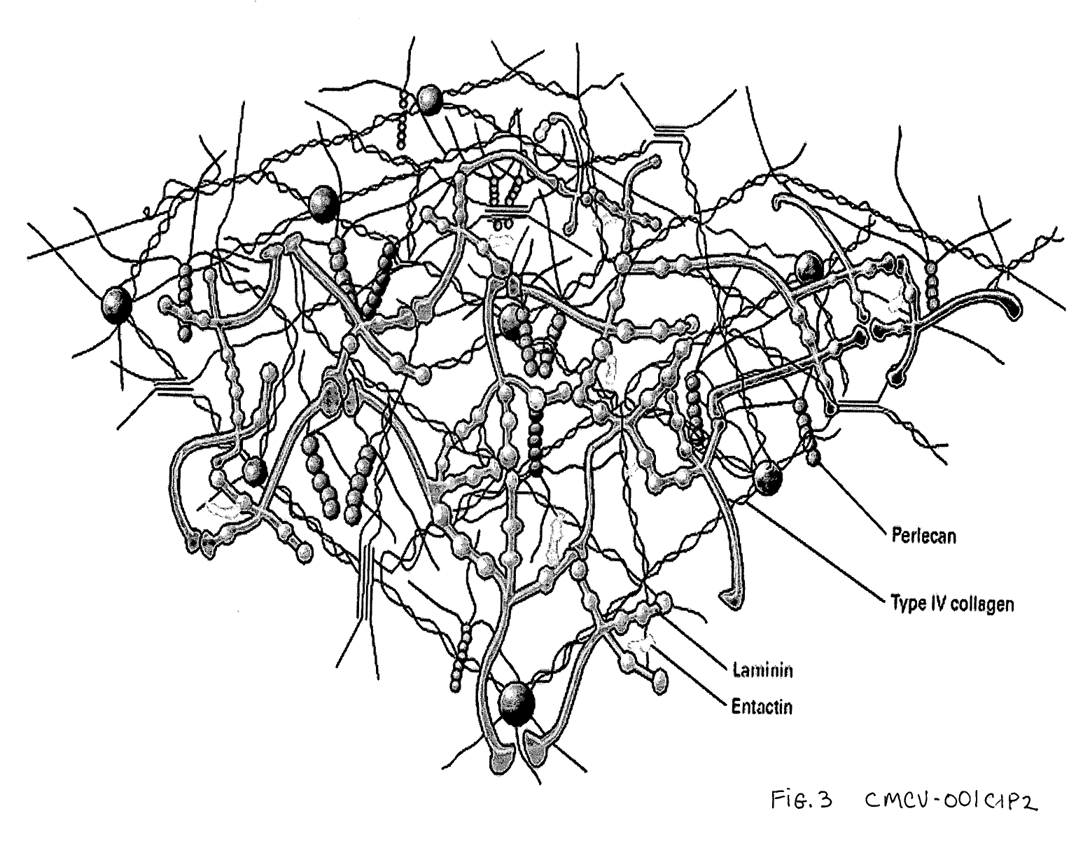

### DETAILED DESCRIPTION OF THE INVENTION

Introduction

Reconstruction, replacement or repair of damaged or defective intracardiac tissue typically requires open heart surgery, and where a prosthetic device or material is used, often (especially in the case of children and valve replacements for adults) second or subsequent open heart surgical procedures are necessary to continually revise upon and update the prosthetic device or material. The present invention provides compositions and methods that may provide the option for a single open heart surgical procedure without the need for second or subsequent procedures, or in some cases, depending on the composition used, may provide the opportunity to address the defect with a single minimally invasive surgical procedure alone. Intracardiac defects include valve defects, congential defects in the intracardium, and any other defect of the intracardium known in the art and identified as a defect in intracardial tissue and presently treated or treatable using prosthetic material or devices. Specific abnormalities of intracardiac tissue include, for example damage to the intracardiac tissue due to trauma or aging, and defects in the intracardiac tissue, for example congential defects, or developmental defects. Some specific abnormalities in intracardiac include, but are not limited to, atrial septal defects, ventricular septal defects, right ventricular outflow, ventricular aneurism repair, patent foramen ovale, and other defects that occur either congentially, or with aging or damage to the tissue.

One such category of damage or defect to intracardiac tissue involves damage or defects of tissue in the conduction pathway of the heart. The conduction pathway runs from the sino atrial node to the atrio ventricular node. Damage or defect can occur at either node or in the pathway that runs between them. The result of damage or defect in the SA or AV nodes of the heart or in the conduction pathway of the heart is inadequate, inefficient, or insufficient electrical impulses. The compositions of the invention can be applied to (e.g. placed at or put in contact with or affixed to) either the SA or AV nodes or to tissue in the conduction pathway in order to restore the electrical conduction to the damaged or defective intracardiac tissue that is causing the failure of the electrical conduction of the heart.

The invention is to a composition comprising extracellular matrix. The composition can be, for example, a patch, an emulsion, an injectable solution, a gel, a fluid, a paste, a powder, a strand, a strip, a spray, a vapor, a cream, or a coating. The composition can further comprise one or more additional components, including, for example, a cell, peptide, polypeptide, protein or other biological moieties. The additional moieties can be in a form such as, for example an emulsion, an injectable solution, a gel, a fluid, a paste, a powder, a strand, a strip, a spray, a vapor, a cream, or a coating. Where the composition is a patch, and the patch can be in a form selected from the group consisting of a sheet, a laminate, a weave, a polymer matrix, a plurality of strands, or one or more strips.

Extracellular matrix materials act as a natural scaffold for repairing soft tissues in the body. Animal studies have shown that the original extracellular matrix material remodels and is replaced by host tissue. Extracellular matrix (for example small intestinal submucosa or SIS) is a resorbable biomaterial which has been used successfully as a xenogenic tissue graft that induces constructive remodeling of a variety of animal tissues including blood vessels, urinary bladder, dura, abdominal wall, tendons and ligaments. The remodeling process includes rapid neovascularization and abundant accumulation of mesenchymal and epithelial cells that support extensive deposition of a new extracellular matrix. Two studies have demonstrated that the noncollagenous portion of the SIS extracellular matrix is composed of various glycoproteins, such as hyluronic acid, heparin, dermatan and chondroitin sulfate A, as well as FGF-2 and TGF-β growth factors.

After processing, the extracellular matrix retains many of the endogenous proteins which act as growth and differentiation factors. These factors stimulate the local environment to populate the extracellular matrix with cells that are then able to differentiate into the original tissue that the extracellular matrix is replacing. Research in rodents has shown that these materials attract pluripotential, marrow derived cells from the animal to regenerate and replace the tissue in a given location. A intracardiac patch of extracellular matrix will act as a mechanical scaffold while the body recruits the necessary cells to remodel and repair the intracardiac tissue.

There has been much research recently to elucidate the properties and function of the extracellular matrix: its protein make-up, and its role in the body. The extracellular matrix (extracellular matrix) is a scaffold matrix of polymerized “structural” proteins that fit into three groups: collagens, glycoproteins, and proteoglycans (which have glycosaminoglycan repeats throughout). These molecules actually polymerize to form the scaffold or matrix of proteins that exists in dynamic interaction with cells, and closely placed functional proteins (either on the cells, or bound to a structural protein). Thus the extracellular matrix also includes within its matrix scaffold “functional” proteins that interact with the structural proteins and with migrating or recruited cells, particularly stem cells in tissue regeneration. The matrix functional proteins also interact with protein expressing cells during the life and maintenance of the matrix scaffold itself as it rebuilds and maintains its components. Note that some proteins fall into both a structural protein classification and a functional protein classification, depending on the protein's configuration and placement in the whole matrix.

The extracellular matrix of cardiac tissue is made up of collagen types I (which is predominant), III, IV, V, and VI, combined which are 92% of the dry weight of the matrix. Glycosaminoglycans (GAGs) include chondroitin sulfate A and B, heparan, heparin, and hyaluronic acid. Glycoproteins such as fibronectin and entactin, proteoglycans such as decorin and perlecan, and growth factors such as transforming growth factor beta (TGF-beta), fibroblast growth factor-2 (FGF-2) and vascular endothelial growth factor (VEGF), are key players in the activity of a myocardium regenerating matrix. Furthermore, the precise chemical constitution of the matrix appears to play a role in its function, including for example what collagen type is prevalent in the matrix, the pore size established by the matrix scaffold, the forces transmitted to adhesion molecules and mechanoreceptors in the cell membranes of cells at the matrix, and the forces directed from the three-dimensional environment (for example the gene expression in the three-dimensional matrix scaffold environment is very different than in a monolayer environment). Thus, the outcome of any tissue regenerative processes is determined by the structural and functional components of the matrix scaffold that form the basis of the regenerative process.

More specifically, when in early regenerative processes, circulating cells or added cells are directed, initial temporary cell adhesion processes occur that result in embryogenesis of the cells, morphogenesis of the cells, regeneration of cell form, eventual maintenance of the cell, possible motility to another site, and organogenesis that further differentiates the cell. Facilitating these early cell adhesion functions are cell adhesion molecules (CAMs). The CAMs are available either endogenously, or added as an additional component of the composition. CAMs are glycoproteins lodged in the surface of the cell membrane or transmembrane connected to cytoskeletal components of the cell. Specific CAMs include cadherins that are calcium dependent, and more than 30 types are known. Also working as CAMs are integrins which are proteins that link the cytoskeleton of the cell in which they are lodged to the extracellular matrix or to other cells through alpha and beta transmembrane subunits on the integrin protein. Cell migration, embryogenesis, hemostatis, and wound healing are so facilitated by the integrins in the matrix. Syndecans are proteoglycans that combine with ligands for initiating cell motility and differentiation. Immunoglobins provide any necessary immune and inflammatory responses. Selectins promote cell-cell interactions.

Specific requirements for the scaffold component of the invention, whether a native scaffold prepared for introduction into a mammal, or a synthetic scaffold formed by synthetic polymerizing molecules, or a combination of the two, are that the scaffold must be resorbable over time as the tissue regeneration ensues, and this resorbtion is at an appropriate degradation rate for optimal tissue regeneration and absence of scar tissue formation. The extracellular matrix scaffold must also be non-toxic, and provide a three-dimensional construction. The matrix scaffold is required to have a high surface area so that there is plenty of room for the biological activities required of the tissue regeneration process. The scaffold must be able to provide cellular signals such as those mentioned herein that facilitate tissue regeneration. Finally the scaffold needs to be non-immunogenic so that it is not rejected by the host, and it needs to be non-thrombogenic.

Particular study of the components of the native scaffolds facilitates design of compositions well-suited for regeneration of intracardium.

The Structural Proteins of the Extracellular Matrix Scaffold

Collagens, the most abundant components of extracellular matrix, are homo- or heterotrimeric molecules whose subunits, the alpha chains, are distinct gene products. To date 34 different alpha chains have been identified. The sequence of the alpha chains contains a variable number of classical Gly-X-Y repetitive motifs which form the collagenous domains and noncollagenous domains. The collagenous portions of 3 homologous or heterologous alpha chains are folded together into a helix with a coiled coil conformation that constitutes the basic structure motif of collagens.

Characteristically, collagens form highly organized polymers. Two main classes of molecules are formed by collagen polymers: the fibril-forming collagens (collagens type I,II, III, V, and XI) and the non-fibrillar collagens that are a more heterogeneous class. Fibril collagen molecules usually have a single collagenous domain repeated the entire length of the molecule, and non-fibrillar collagen molecules have a mixture of collagenous and noncollagenous domains. On this basis several more subgroups of the collagen family are identified: e.g. the basement membrane collagens (IV, VIII, and X). In addition, most all the different types of collagen have a specific distribution. For example, fibril forming collagens are expressed in the interstitial connective tissue. The most abundant component of basement membranes is collagen IV. The multiplexins, collagens XV and XVIII are also localized to the basement membranes.

In the extracellular matrix of the heart, collagen types I and III predominate, together forming fibrils and providing most of the connective material for typing together myocytes and other structures in the myocardium, and thus these molecule types are involved in the transmission of developed mechanical force in the heart. Only collagen types I, II, III, V, and XI self assemble into fibrils, characterized by a triple helix in the collagen molecules. Some collagens form networks, as with the basement membrane, formed by collagen IV. Type III collagen dominates in the wall of blood vessels and hollow intestinal organs and copolymerizes with type I collagen.

Proteoglycans are grouped into several families, and all have a protein core rich in glycosoaminoglycans. They control proliferation, differentiation, and motility. The lecticans interact with hyaluronan and include aggrecan, versican, neurocan, and brevican. Versican stimulates proliferation of fibroblasts and chondrocytes through the presence in the molecule of EGF-like motifs. The second type of proteoglycans have a protein core with leucine-rich repeats, which form a horse shaped protein good for protein-protein interactions. Their glycosoaminoglycan side chains are mostly chondroitin/dermatan sulphate or keratin sulphate. Decorin, biglycan, fibromodulin, and keratocan are members of this family. Decorin is involved in modulation and differentiation of epithelial and endothelial cells. In addition, transforming growth factor beta (TGF beta) interacts with members of this family. There are part-time proteoglycans, comprising CD44 (a receptor for hyaluronic acid), macrophage colony stimulating factor, amyloid precursor protein and several collagens (IX, XII, XIV, and XVIII). The last family of proteoglycans is the heparan sulfate proteoglycans, some of which are located in the matrix, and some of which are on cell membranes. Perlecan and agrin are matrix heparan sulfate proteoglycans found in basement membranes. The syndecans and glypicans are membrane-associated heparan sulfate proteoglycans. Syndecans have a heparan sulfate extracellular moiety that binds with high affinity cytokines and growth factors, including fibroblast growth factor (FGF), hepatocyte growth factor (HGF), platelet-derived growth factor (PDGF), heparin-binding epidermal growth factor (HB-EGF), and vascular endothelial growth factor (VEGF). The heparan sulfate proteoglycans have been implicated in modulation of cell migration, proliferation and differentiation in wound healing.

Glycoproteins are also structural proteins of extracellular matrix scaffold. The glycoprotein fibronectin (Fn) is a large dimer that attracts stem cells, fibroblasts and endothelial cells to a site of newly forming matrix. Tenascin is a glycoprotein that has Fn repeats and appears during early embryogenesis then is switched off in mature tissue. Tenascin reappears during wound healing. Other glycoprotein components of extracellular matrix include elastin that forms the elastic fibers and is a major structural component along with collagen; fibrillins which are a family of proteins consisting almost entirely of endothelial growth factor (EGF)-like domains. Small glycoproteins present in extracellular matrix include nidogen/entactin and fibulins I and II.

The glycoprotein laminin is a large protein with three distinct polypeptide chains. Together with type IV collagen, nidogen, and perlecan, laminin is one of the main components of the basement membrane. Laminin isoforms are synthesized by a wide variety of cells in a tissue-specific manner. Laminin I contains multiple binding sites to cellular proteins. Virtually all epithelial cells synthesize laminin, as do small, skeletal, and cardiac muscle, nerves, endothelial cells, bone marrow cells, and neuroretina. Laminins affect nearby cells, by promoting adhesion, cell migration, and cell differentiation. They exert their effects mostly through binding to integrins on cell surfaces. Laminins 5 and 10 occur predominantly in the vascular basement membrane and mediate adhesion of platelets, leukocytes, and endothelial cells.

The Functional Proteins of the Extracellular Matrix Scaffold

In addition to the structural matrix proteins just discussed, specific interactions between cells and the extracellular matrix are mediated by functional proteins of the extracellular matrix, including transmembrane molecules, mainly integrins, some members of the collagen family, some proteoglycans, glycosaminoglycan chains, and some cell-surface associated proteins. These interactions lead to direct or indirect control of cellular activities within the extracellular matrix scaffold such as adhesion, migration, differentiation, proliferation, and apoptosis.

Glycosaminoglycans (GAGs) are glycosylated post-translational molecules derived from proteoglycans. Well known GAGs include heparin, hyaluronic acid, heparan sulfate, and chondroitin sulfate A, B, and C. Heparin chains stimulate angiogenesis, and act as subunits in a proteoglycan to stimulate the angiogenic effects of fibroblast growth factor-2 (FGF-2) (also known as basic FGF or bFGF). Chondroitin sulfate B (dermatan sulfate) interacts with TGF-beta to control matrix formation and remodeling. The proteoglycan form of chondroitin sulfate B regulates the structure of extracellular matrix by controlling collagen fibril size, orientation and deposition. Hyaluronic acid is associated with rapid wound healing and organized deposit of collagen molecules in the matrix. It is believed that hyaluronic acid binds TGF-betal to inhibit scar formation.

The extracellular matrix is also being remodeled constantly in the live animal. The proteins of the extracellular matrix are broken down by matrix metalloproteases, and new protein is made and deposited as replacement protein. Collagens are mostly synthesized by the cells comprising the extracellular matrix: fibroblasts, myofibroblasts, osteoblasts, and chondrocytes. Some collagens are also synthesized by adjacent parenchymal cells or also covering cells such as epithelial, endothelial, or mesothelial cells.

The extracellular part of integrins bind fibronectin, collagen and laminin, and act primarily as adhesion molecules. Integrin-ligand binding also triggers cascades of activity for cell survival, cell proliferation, cell motility, and gene transcription.

Tenascins include cytotactin (TN-C). Cell surface receptors for tenascins include integrins, cell adhesion molecules of the Ig superfamily, a transmembrane chrondroitin sulfate proteoglycan (phosphacan) and annexin II. TN-C also interacts with extracellular proteins such as fibronectin and the lecticans (the class of extracellular chondroitin sulphate proteoglycans including aggrecan, versican, and brevican).

In addition to direct knowledge of protein cell interaction many of the proteins associated with the extracellular matrix can initiate binding to proteins that then activate to bind other proteins or cells, e.g. decorin binds Fn or thrombospondin and causes their cell adhesion promoting activity. Other proteoglycans control the hydration of the extracellular matrix and the spacing between the collagen fibrils and network, which is believed to facilitate cell migration. Proteoglycans regulate cell function by controlling growth factor activity, e.g. decorin, biglycan, and fibromodulin bind to isoforms of transforming growth factor beta (TGF beta) and heparin sulfate proteoglycans bind and store fibroblast growth factor.

The matrix metalloproteases (MMPs) break down the collagen molecules in the extracellular matrix so that new collagen can be used to remodel and renew the extracellular matrix scaffold. It is also believed that the proteolytic activity of MMPs augment the bioavailability of growth factors sequestered within the extracellular matrix, and can activate latent secreted growth factors like TGF-beta and IGF from IGFBPs and cell surface growth factor precursors. MMPs can proteolytically cleave cell surface growth factors, cytokines, chemokine receptors and adhesion receptors, and thus participate in controlling responses to growth factors, cytokines, chemokines, as well as cell-cell and cell-extracellular matrix interactions.

Structural or functional matrix proteins that can comprise the compositions herein disclosed to facilitate myocardial tissue regeneration include, minimally, collagen I and III, elastin, laminin, CD44, hyaluronan, syndecan, bFGF, HGF, PDGF, VEGF, Fn, tenascin, heparin, heparan sulfate, chondroitin sulfate B, integrins, decorin, and TGF-beta.

Native Sources and Preparations

Native extracellular matrix scaffolds, and the proteins that form them, are found in their natural environment, the extracellular matrices of mammals. These materials are prepared for use in mammals in tissue graft procedures. Small intestine submucosa (SIS) is described in U.S. Pat. No. 5,275,826, urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710, to name some of the extracellular matrix scaffolds presently available for explanting procedures. In addition, collagen from mammalian sources can be retrieved from matrix containing tissues and used to form a matrix composition. Extracellular matrices can be synthesized from cell cultures as in the product manufactured by Matrigel™. In addition, dermal extracellular matrix material, subcutaneous extracellular matrix material, large intestine extracellular matrix material, placental extracellular matrix material, omamentum extracellular matrix material, heart extracellular matrix material, and lung extracellular matrix material, may be used, derived and preserved similarly as described herein for the SIS, SS, LBM, and UBM materials. Other organ tissue sources of basement membrane for use in accordance with this invention include spleen, lymph nodes, salivary glands, prostate, pancreas and other secreting glands. In general, any tissue of a mammal that has an extracellular matrix can be used for developing an extracellular matrix component of the invention.

When using collagen-based synthetic extracellular matrices, the collagenous matrix can be selected from a variety of commercially available collagen matrices or can be prepared from a wide variety of natural sources of collagen. Collagenous matrix for use in accordance with the present invention comprises highly conserved collagens, glycoproteins, proteoglycans, and glycosaminoglycans in their natural configuration and natural concentration. Collagens can be from animal sources, from plant sources, or from synthetic sources, all of which are available and standard in the art.

The proportion of scaffold material in the composition when native scaffold used will be large, as the natural balance of extracellular matrix proteins in the native scaffolds usually represents greater than 90% of the extracellular matrix material by dry weight. Accordingly, for a functional tissue regenerative product, the scaffold component of the composition by weight will be generally greater than 50% of the total dry weight of the composition. Most typically, the scaffold will comprise an amount of the composition by weight greater than 60%, greater than 70%, greater than 80%, greater than 82%, greater than 84%, greater than 86%, greater than 88%, greater than 90%, greater than 92%, greater than 94%, greater than 96%, and greater than 98% of the total composition.

Native extracellular matrices are prepared with care that their bioactivity for myocardial tissue regeneration is preserved to the greatest extent possible. Key functions that may need to be preserved include control or initiation of cell adhesion, cell migration, cell differentiation, cell proliferation, cell death (apoptosis), stimulation of angiogenesis, proteolytic activity, enzymatic activity, cell motility, protein and cell modulation, activation of transcriptional events, provision for translation events, inhibition of some bioactivities, for example inhibition of coagulation, stem cell attraction, and chemotaxis. Assays for determining these activities are standard in the art. For example, material analysis can be used to identify the molecules present in the material composition. Also, in vitro cell adhesion tests can be conducted to make sure that the fabric or composition is capable of cell adhesion.

The matrices are generally decellularized in order to render them non-immunogenic. A critical aspect of the decellularization process is that the process be completed with some of the key protein function retained, either by replacement of proteins incidentally extracted with the cells, or by adding exogenous cells to the matrix composition after cell extraction, which cells produce or carry proteins needed for the function of tissue regeneration in vivo.

Prudent practice may dictate that the cell extract from the patches be tested for its protein make-up, so that if necessary proteins are removed they can be place back into the matrix composition, perhaps using exogenous proteins at approximately the same amount as those detected in the extraction solution. Another option would be that the proteins extracted during the cell extraction process can simply be added back after the cell extraction is complete, thus preserving the desired bioactivity in the material.

The bioactivity of extracellular matrix material can be mimicked in tissue regeneration experiments with combinations of native and synthetic extracellular matrices explanted together, also optionally with additional components such as proteins or cells, in order to provide an optimal intracardiac tissue regenerative composition and environment in vivo. What works as the best composition for intracardial tissue regeneration in patients, particularly humans can be tested first in other mammals by standard explanting procedures to determine whether tissue regeneration is accomplished and optimized by a particular composition. See Badylak et al, The Heart Surgery Forum, Extracellular Matrix for Myocardial Repair 6(2) E20-E26 (2003).

When adding proteins to the extracellular matrix composition, the proteins may be simply added with the composition, or each protein may be covalently linked to a molecule in the matrix. Standard protein-molecule linking procedures may be used to accomplish the covalent attachment.

For decellularization when starting with a whole organ as a source of mammalian extracellular matrix, whole organ perfusion process can be used. The organ is perfused with a decellularization agent, for example 0.1% peractic acid rendering the organ acellular. The organ can then be cut into portions and stored (e.g. in aqueous environment, liguid nitrogen, cold, freeze-dried, or vacuum-pressed) for later use. Any appropriate decellularizing agent may be used in whole organ perfusion process.

With regard to submucosal tissue, extractions may be carried out a near neutral pH (in a range from about pH 5.5 to about pH 7.5) in order to preserve the presence of growth factor in the matrices. Alternatively, acidic conditions (i.e. less than 5.5 pH) can be used to preserved the presence of glycosaminoglycan components, at a temperature in a range between 0 and 50 degrees centrigrade. In order to regulate the acidic or basic environment for these aqueous extractions, a buffer and chaotropic agent (generally at a concentration from about 2M to about 8M) are selected, such as urea (at a concentration from about 2M to 4M), guanidine (at a concentration from about 2M to about 6M, most typically about 4M), sodium chloride, magnesium chloride, and non-ionic or ionic surfactants. Urea at 2M in pH 7.4 provides extraction of basis FGF and the glycoprotein fibronectin. Using 4M guanidine with pH 7.4 buffer yields a fraction having transforming growth factor beta. (TGF-beta). Accordingly, it may behoove a practitioner to decellularize one portion of a matrix, and extract desired proteins to add back in from other different portions.

Because of the collagenous structure of basement membrane and the desire to minimize degradation of the membrane structure during cell dissociation, collagen specific enzyme activity should be minimized in the enzyme solutions used in the cell-dissociation step. For example, liver tissue is typically also treated with a calcium chelating agent or chaotropic agent such as a mild detergent such as Triton 100. The cell dissociation step can also be conducted using a calcium chelating agent or chaotropic agent in the absence of an enzymatic treatment of the tissue. The cell-dissociation step can be carried out by suspending liver tissue slices in an agitated solution containing about 0.05 to about 2%, more typically about 0.1 to about 1% by weight protease, optionally containing a chaotropic agent or a calcium chelating agent in an amount effective to optimize release and separation of cells from the basement membrane without substantial degradation of the membrane matrix.

After contacting the liver tissue with the cell-dissociation solution for a time sufficient to release all cells from the matrix, the resulting liver basement membrane is rinsed one or more times with saline and optionally stored in a frozen hydrated state or a partially dehydrated state until used as described below. The cell-dissociation step may require several treatments with the cell-dissociation solution to release substantially all cells from the basement membrane. The liver tissue can be treated with a protease solution to remove the component cells, and the resulting extracellular matrix material is further treated to remove or inhibit any residual enzyme activity. For example, the resulting basement membrane can be heated or treated with one or more protease inhibitors.

Basement membrane or other native extracellular matrix scaffolds may be sterilized using conventional sterilization techniques including tanning with glutaraldehyde, formaldehyde tanning at acidic pH, ethylene oxide treatment, propylene oxide treatment, gas plasma sterilization, gamma radiation, and peracetic acid sterilization. A sterilization technique which does not significantly weaken the mechanical strength and biotropic properties of the material is preferably used. For instance, it is believed that strong gamma radiation may cause loss of strength in the graft material. Preferred sterilization techniques include exposing the graft to peracetic acid, low dose gamma irradiation and gas plasma sterilization; peracetic acid sterilization being the most preferred method.

Synthetic

Synthetic extracellular matrices can be formed using synthetic molecules that polymerize much like native collagen and which form a scaffold environment that mimics the native environment of mammalian extracellular matrix scaffolds. According, such materials as polyethylene terephthalate fiber (Dacron), polytetrafluoroethylene (PTFE), glutaraldehyde-cross linked pericardium, polylactate (PLA), polyglycol (PGA), hyaluronic acid, polyethylene glycol (PEG), polyethelene, nitinol, and collagen from non-animal sources (such as plants or synthetic collagens), can be used as components of a synthetic extracellular matrix scaffold. The synthetic materials listed are standard in the art, and forming hydrogels and matrix-like materials with them is also standard. Their effectiveness can be tested in vivo as sited earlier, by testing in mammals, along with components that typically constitute native extracellular matrixs, particularly the growth factors and cells responsive to them.

The extracellular matrix-like materials are described generally in the review article “From Cell-extracellular matrix Interactions to Tissue Engineering” Rosso et al, Journal of Cellular Physiology 199:174-180 (2004). In addition, some extracellular matrix-like materials are listed here. Particularly useful biodegradable and/or bioabsorbable polymers include polylactides, poly-glycolides, polycarprolactone, polydioxane and their random and block copolymers. Examples of specific polymers include poly D,L-lactide, polylactide-co-glycolide (85:15) and polylactide-co-glycolide (75:25). Preferably, the biodegradable and/or bioabsorbable polymers used in the fibrous matrix of the present invention will have a molecular weight in the range of about 1,000 to about 8,000,000 g/mole, more preferably about 4,000 to about 250,000 g/mole. The biodegradable and/or bioabsorbable fiberizable material is preferably a biodegradable and bioabsorbable polymer. Examples of suitable polymers can be found in Bezwada, Rao S. et al. (1997) Poly(p-Dioxanone) and its copolymers, in Handbook of Biodegradable Polymers, A. J. Domb, J. Kost and D. M. Wiseman, editors, Hardwood Academic Publishers, The Netherlands, pp. 29-61. The biodegradable and/or bioabsorbable polymer can contain a monomer selected from the group consisting of a glycolide, lactide, dioxanone, caprolactone, trimethylene carbonate, ethylene glycol and lysine. The material can be a random copolymer, block copolymer or blend of monomers, homopolymers, copolymers, and/or heteropolymers that contain these monomers. The biodegradable and/or bioabsorbable polymers can contain bioabsorbable and biodegradable linear aliphatic polyesters such as polyglycolide (PGA) and its random copolymer poly(glycolide-co-lactide-) (PGA-co-PLA). The FDA has approved these polymers for use in surgical applications, including medical sutures. An advantage of these synthetic absorbable materials is their degradability by simple hydrolysis of the ester backbone in aqueous environments, such as body fluids. The degradation products are ultimately metabolized to carbon dioxide and water or can be excreted via the kidney. These polymers are very different from cellulose based materials, which cannot be absorbed by the body.

Other examples of suitable biocompatible polymers are polyhydroxyalkyl methacrylates including ethylmethacrylate, and hydrogels such as polyvinylpyrrolidone, polyacrylamides, etc. Other suitable bioabsorbable materials are biopolymers which include collagen, gelatin, alginic acid, chitin, chitosan, fibrin, hyaluronic acid, dextran, polyamino acids, polylysine and copolymers of these materials. Any glycosaminoglycan (GAG) type polymer can be used. GAGs can include, e.g., heparin, chondroitin sulfate A or B, and hyaluronic acid, or their synthetic analogues. Any combination, copolymer, polymer or blend thereof of the above examples is contemplated for use according to the present invention. Such bioabsorbable materials may be prepared by known methods.

Nucleic acids from any source can be used as a polymeric biomaterial. Sources include naturally occurring nucleic acids as well as synthesized nucleic acids. Nucleic acids suitable for use in the present invention include naturally occurring forms of nucleic acids, such as DNA (including the A, B and Z structures), RNA (including mRNA, tRNA, and rRNA together or separated) and cDNA, as well as any synthetic or artificial forms of polynucleotides. The nucleic acids used in the present invention may be modified in a variety of ways, including by cross linking, intra-chain modifications such as methylation and capping, and by copolymerization. Additionally, other beneficial molecules may be attached to the nucleic acid chains. The nucleic acids may have naturally occurring sequences or artificial sequences. The sequence of the nucleic acid may be irrelevant for many aspects of the present invention. However, special sequences may be used to prevent any significant effects due to the information coding properties of nucleic acids, to elicit particular cellular responses or to govern the physical structure of the molecule. Nucleic acids may be used in a variety of crystalline structures both in finished biomaterials and during their production processes. Nucleic acid crystalline structure may be influenced by salts used with the nucleic acid. For example, Na, K, Bi and Ca salts of DNA all have different precipitation rates and different crystalline structures. Additionally, pH influences crystalline structure of nucleic acids.

The physical properties of the nucleic acids may also be influenced by the presence of other physical characteristics. For instance, inclusion of hairpin loops may result in more elastic biomaterials or may provide specific cleavage sites. The nucleic acid polymers and copolymers produced may be used for a variety of tissue engineering applications including to increase tissue tensile strength, improve wound healing, speed up wound healing, as templates for tissue formation, to guide tissue formation, to stimulate nerve growth, to improve vascularization in tissues, as a biodegradable adhesive, as device or implant coating, or to improve the function of a tissue or body part. The polymers may also more specifically be used as sutures, scaffolds and wound dressings. The type of nucleic acid polymer or copolymer used may affect the resulting chemical and physical structure of the polymeric biomaterial.

Additional Components: Cells

Unlike skeletal myocytes, cardiomyocytes withdraw from cell cycle shortly after birth, and adult mammalian cardiomyocytes lack the potential to proliferate. Therefore, in order to regenerate the intracardium, the right cells may have to be added to the composition, or the site, or the right molecules to attract the right cells will have to be added to the composition or the site. Transplantation cell sources for the intracardiac tissue include allogenic, xenogenic, or autogenic sources. Accordingly, human embryonic stem cells, neonatal cardiomyocytes, myofibroblasts, mesenchymal cells, autotransplanted expanded cardiomyocytes, and adipocytes can be used as additive components to accompany the scaffold.

Embryonic stem cells begin as totipotent cells, differentiate to pluripotent cells, and then further specialization. They are cultured ex vivo and in the culture dish environment differentiate either directly to heart muscle cells, or to bone marrow cells that can become heart muscle cells. The cultured cells are then transplanted into the mammal, either with the composition or in contact with the scaffold and other components.

Myoblasts are another type of cell that lend themselves to transplantation into intracardium, however, they do not always develop into cardiomyocytes in vivo. Adult stem cells are yet another species of cell that work in the context of tissue regeneration. Adult stem cells are thought to work by generating other stem cells (for example those appropriate to myocardium) in a new site, or they differentiate directly to a cardiomyocyte in vivo. They may also differentiate into other lineages after introduction to organs, such as the heart. The adult mammal provides sources for adult stem cells in circulating endothelial precursor cells, bone marrow-derived cells, adipose tissue, or cells from a specific organ. It is known that mononuclear cells isolated from bone marrow aspirate differentiate into endothelial cells in vitro and are detected in newly formed blood vessels after intramuscular injection. Thus, use of cells from bone marrow aspirate may yield endothelial cells in vivo as a component of the composition.

Yet another viable option for cells to use in the invention is the mesenchymal stem cells administered with activating cytokines. Subpopulations of mesenchymal cells have been shown to differentiate toward myogenic cell lines when exposed to cytokines in vitro.

Once a type of cell is chosen, the number of cells needed is determined. Their function and anticipated change upon implantation, as well as their viability during the process of transplantation need to be considered to determine the number of cells to transplant. Also the mode of transplantation is to be considered: several modes including intracoronary, retrograde venous, transvascular injection, direct placement at the site, thoracoscopic injection and intravenous injection can be used to put the cells at the site or to incorporate them with the composition either before delivery or after delivery to the defective myocardium. In all cases, the mode of delivery and whether the cells are first mixed with the other components of the composition is a decision made based on what will provide the best chance for viability of the cells, and the best opportunity for their continued development into cells that can function in the scaffold in vivo in order to signal and promote tissue regeneration.

The following list includes some of the cells that may be used as additional cellular components of the composition of the invention: a human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell.

In particular, human embryonic stem cells, fetal cardiomyoctes, mesenchymal stem cells, adipocytes, bone marrow progenitor cells, embryonic stem cells, adult stem cells, or post-natal stem cells together with growth factors or alone with matrix scaffold optimize intracardium regeneration in vivo.

Cells can be seeded directly onto matrix scaffold sheets under conditions conducive to eukaryotic cell proliferation. The highly porous nature of extracellular matrices in particular will allow diffusion of cell nutrients throughout the membrane matrix. Thus, cells can be cultured on or within the matrix scaffold itself. With the emulsified extracellular matrix compositions, or with some of the other formulations, the cells can be co-cultured with the extracellular matrix material before administration of the complete composition to the patient.

Additional Components: Peptides, Polypeptides, or Proteins

In addition to a native extracellular matrix scaffold, or a synthetic scaffold, or a mixture of the two, peptides, polypeptides or proteins can be added. Such components include extracellular structural and functional proteins in admixture so as to mimic either heart extracellular matrix, or other native extracellular matrices that are capable of regenerating at least some reasonable percentage of the defective intracardium, for example at least 30%, preferably more than 50%. Effective regeneration of the intracardium relies on the extracellular matrix scaffold by its structure and components. Mimicking the native explant material as closely as possible thus optimizes the opportunity for regeneration using a composition comprising some native extracellular matrix, albeit treated, but also with additional components.

The peptides, polypeptides or proteins that can be added to the scaffold are: a collagen, a proteoglycan, a glycosaminoglycan (GAG) chain, a glycoprotein, a growth factor, a cytokine, a cell-surface associated protein, a cell adhesion molecule (CAM), an angiogenic growth factor, an endothelial ligand, a matrikine, a matrix metalloprotease, a cadherin, an immunoglobin, a fibril collagen, a non-fibrillar collagen, a basement membrane collagen, a multiplexin, a small-leucine rich proteoglycan, decorin, biglycan, a fibromodulin, keratocan, lumican, epiphycan, a heparan sulfate proteoglycan, perlecan, agrin, testican, syndecan, glypican, serglycin, selectin, a lectican, aggrecan, versican, nuerocan, brevican, cytoplasmic domain-44 (CD-44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), and vascular epithelial growth factor (VEGF).

Typically, the additional peptide, polypeptide, or protein component will comprise an amount of the composition by weight selected from the group consisting of greater than 0.1%, greater than 0.5%, greater than 1%, greater than 1.5%, greater than 2%, greater than 4%, greater than 5%, greater than 10%, greater than 12%, greater than 15%, and greater than 20%.

Whether a particular protein component or combination of components is effective for myocardial tissue regeneration can be tested by contacting the composition with defective myocardium in a test animal, for example a dog, pig, rat or sheep, or other common test mammal. Myocardial tissue regeneration and myocardium contractility are both indicia to measure the success of the composition and procedure, by procedures standard in the art. In addition, a small sampling of the regenerated tissue can be made to determine that new extracellular matrix and new tissue has been made. As to what balance between structural extracellular matrix proteins and functional ones to use in a given composition, nature provides direction. Most extracellular matrixs are predominantly made up of structural proteins by dry weight. Thus only a small portion of functional proteins by weight are needed for effective myocardial tissue regeneration.

Peptides, polypeptides or proteins for the composition may be formulated as is standard in the art for the particular class of protein, and that formulation may be added to the extracellular matrix material (of whatever formulation) for delivery into the patient.

Alternatively, the protein molecules may be covalently linked to an appropriate matrix molecule of any of the matrix formulations. Covalent linking of the protein molecules to molecules of the matrix may be accomplished by standard covalent linking methods known in the art.

Additional Components: Vector Expressing DNA, Nutrients, Drug Molecules

Some of the proteins required for the composition can be genetically synthesized in vivo with DNA and vector constituents. Thus a vector having a DNA capable of targeted expression of a selected gene can contribute a bioactive peptide, polypeptide, or protein to the composition. Standard in vivo vector gene expression can be employed.

In addition, other additives such as a nutrient, a sugar, a fat, a lipid, an amino acid, a nucleic acid, a ribo-nucleic acid, may provide support to the regenerative process in vivo in the composition. Finally, also a drug, such as a heart regenerating or angiogenesis promoting drug may be also added to the composition, in such a form as, for example, an organic molecule, an inorganic molecule, a small molecule, a drug, or any other drug-like bioactive molecule.

Methods of Use

Tissue growth and contractility of extracellular matrix can be tested and observed by standard means, for example as described in Badylak et al, The Heart Surgery Forum, Extracellular Matrix for Myocardial Repair 6(2) E20-E26 (2003).

The invention includes a method of reconstruction, replacement or repair of a defect in intracardiac tissue, comprising contacting the defect with the composition described herein. Contacting can comprise surgically opening the patient at the site of defect and placing the composition at the site. Placing comprises affixing a patch to the intracardiac tissue, for example by affixing the patch, further by example using a means including, but not limited to, suturing, stapling, gluing, or tying the patch to intracardiac tissue. Standard means for affixing prosthetic material patches can be used to affix the patch to the intracardial tissue.

Contacting the defect in intracardiac tissue with the composition can also comprise delivering the composition to the site of defect in the intracardiac tissue using a catheter in a minimally invasive procedure. Contacting can also comprise delivering the composition to the site of defect in the intracardiac tissue using a means such as, for example, but not limited to, injecting, injecting with a percutaneous catheter, CO₂ powering emission of fine emulsion, micronizing an injectable solution, or ink jet delivery. The means for delivery of the composition in a minimally invasive procedure is tailored to the form of the composition itself. For example, generally an emulsion or liquid injectable solution can be delivered by percutanous catheter directly to the site of defect of the intracardiac tissue. Minimally invasive procedures to the heart using percutaneous catheters or other minimally invasive delivery devices are well known in the art. Procedures for accessing the heart using minimally invasive catheters or other delivery devices of injectable materials are well known in the art. The heart is accessed with a catheter that is loaded with sufficient injectable material. Visualization techniques common in the art for locating and directing catheter or device positioning are used to locate the tip of the catheter at the site of damage or defect where the injectable or emulsion should be delivered. The injectable or emulsion is delivered to the site when it is deemed that the catheter or device tip is in the proper location. It may be necessary to spread the material over the surface of the tissue that is being treated, which can be accomplished with the tip of the catheter or other delivery device. Once in place and after is it determined that sufficient material has been delivered to effect a repair, the device can be withdrawn from the site. Repair of the tissue can be monitored by visualization during subsequent time periods to ensure that the procedure has been effective in achieving reconstruction or repair of the damaged or defective intracardiac tissue.

The invention further provides a method of making a composition to address a defect in intracardiac tissue. The method of making comprises preparing the composition, and packaging the composition for storage until use. Preparing extracellular matrix compositions are described herein, and storage of the same is standard in the art of extracellular matrix.

Kits

A kit for sale of a composition comprising extracellular matrix can be assembled. Where the composition is a patch the kit can be offered with several size options, containing, for example, patches of all the same sizes, or a combination of sizes. Where the composition is in an alternative form, for example, an emulsion, an injectable solution, a gel, a fluid, a paste, a powder, a strand, a strip, a spray, a vapor, a cream, or a coating, the composition is packaged as is appropriate for the form of the material, using standard procedures known in the art. In any event, directions for use and application of the composition are included in the kit, including how the composition is to be affixed or delivered to the site of defect in the intracardiac tissue. The kit can also include items to accomplish one or more means to affix or deliver the composition to the site of defect in intracardiac issue. For example, for affixing a patch, the kit can include, suture, glue, staples or ties for example. For delivering an emulsion or liquid injectable composition, the kit may include a delivery device, for example a catheter. The catheter can be pre-loaded with the composition. The kit also provides a container for holding the contents of the kit.

The extracellular matrix composition, if derived from a mammal, can include e.g. SIS, UBS, SS, LS, LBM, or UBM (urinary basement membrane), or other native extracellular matrices. If the composition is a patch comprising synthetic extracellular matrix, the patch or patches in the kit can comprise a weave of strands, for example a weave of native and synthetic strands. The native or synthetic strands can be made from, for example but not limited to, mammalian extracellular matrix, synthetic extracellular matrix, polymer, plastic, metal, metal alloy, Dacron, or nylon. The strands are generally biocompatible, and some may or may not be bioabsorbable.

### EXAMPLES

### Example 1

A intracardiac patch is made of extracellular matrix scaffold derived from porcine small intestinal submucosa (SIS). SIS was developed from a select layer of tissue that is recovered from porcine small intestine. During processing, the inner and outer muscle layers of the material are removed, leaving an intact submucosa with a portion of the tunica propria layer attached to the outer surface. Following processing, the remaining acellular extracellular material is cut to specific shapes and sizes, lyophilized, and terminally sterilized using ethylene oxide gas. The intracardiac patch is supplied in four-ply sheets of various dimensions, which can be cut to size as the physician deems necessary for the procedure. The patch is provided to the customer in the lyophilized, sterile state. The patch product is sterilized by ethylene oxide (EtO). The patch can be packaged in a sterile, double, tyvek pouch and then placed inside a paperboard box for shipment to the customer.

### Example 2

The intracardiac patch is tested for viral inactivation. Viral inactivation studies are performed to assess the safety and effectiveness of the device. Viral Inactivation Testing is performed in accordance with the Good Laboratory Practices regulations, 21 CFR Section 58, to validate the inactivation of viral contamination during disinfection processing of the SIS material comprising the percardial patch. The methods used are based on the European Committee for Standardization, prEN12442-3: 1996, Animal tissues and their derivatives utilized in the manufacture of medical devices—Part 3: Validation of the elimination and/or inactivation of viruses and other transmissible agents. Results will demonstrate that the disinfection process reduces viral load to a SAL of at least 10^{31 6}. Inactivation of Spiked Parvovirus and Reovirus During Reduced-Scale Processing/Disinfection of Porcine Small Intestine Sheets; Inactivation of Spike Murine Leukemia Retrovirus and Porcine Pseudorabies (Herpes) Virus During Reduced-Scale Processing/Disinfection of Porcine Small Intestine Sheets; Probe Burst Strength Test of Four-layer, Lyophilized, SIS; Suture Retention Strength of Multilayer (4) Lyophilized SIS; Suture Retention Strength of Multilayer (4) Lyophilized SIS; Tensile Strength and Thickness of 4-layer, Freeze-dried, High Strength, SIS Sheet.

The patch is EtO sterilized to a sterility assurance level of 10⁻⁶. EtO sterilization is considered a traditional method of sterilization for medical devices. The intracardial patch comprising lyophilized sheets can be labeled shelf-life of 18-22 months.

### Example 3

A kit is assembled for sale of the intracardiac patch product to surgeons and hospitals. The kit can contain a selection of intracardiac patch sizes, and for affixing the patch to the intracardiac tissue, the kit can also include materials such as suture, glue, ties, or staples to affix the patch to the site of defect in the intracardiac tissue. Directions in the kit indicate how to affix the patch to the site, or how to otherwise deliver the composition to the site, for example, where the composition is an emulsion or a liquid injectable. The kit can also contain information about how the composition is sterilized, and directions for care of the stored composition, and the estimated shelf-life of the composition

### Example 4

A child is presented with a congenital defect in intracardiac tissue. A composition comprising an emulsion of native extracellular matrix is prepared and a percutanous catheter is loaded with the emulsion. A minimally invasive procedure is conducted to deliver the emulsion to the site of defect in the child, guided by a visualization technic such as radiography following a radio-opaque portion of the catheter to determine that the catheter is positioned to deliver the emulsion to the proper site in the intracardiac tissue. The emulsion is released at the site, and the catheter withdrawn. The catheter entry site is closed and the patient monitored for response to the procedure.

All references cited are incorporated in their entirety. Although the foregoing invention has been described in detail for purposes of clarity of understanding, it will be obvious that certain modifications may be practiced within the scope of the appended claims.

What is claimed is:

1. A composition for reconstruction, replacement or repair of damaged or defective intracardiac tissue comprising at least a portion of an extracellular matrix scaffold component derived from a mammalian or synthetic source and a first additional component selected from the group consisting of a collagen present in myocardial tissue, a collagen type IV protein, a collagen type VIII Protein, a collagen type X protein, a multiplexin, a matrix heparin sulfate protoeglycan, a laminin, a nidogen, an agin, and a perlecan, wherein the composition comprises a form selected from the group consisting of a patch, an emulsion, an injectable solution, a gel, a fluid, a paste, a powder, a strand, a strip, a spray, a vapor, a cream, and a coating.

2. The composition of claim 1 comprising a patch, wherein the patch is in a form selected from the group consisting of a sheet, a laminate, a weave, a polymer matrix, a plurality of strands, and one or more strips.

3. The composition of claim 1, comprising mammalian extracellular matrix scaffold component.

4. The composition of claim 1, comprising synthetic extracellular matrix scaffold component.

5. The composition of claim 1, comprising both mammalian and synthetic extracellular matrix scaffold component.

6. The composition of claim 3, wherein the mammalian extracellular matrix scaffold component comprises extracellular matrix selected from the group consisting of small intestine submucosa (SIS), urinary bladder submucosa (UBS), urinary basement membrane (UBM), liver basement membrane (LBM), stomach submucosa (SS), collagen from animal sources, collagen from plant sources, synthesized extracellular matrix in cultures from cells (Matrigel™), dermal extracellular matrix, subcutaneous extracellular matrix, large intestine extracellular matrix, placental extracellular matrix, ornamentum extracellular matrix, heart extracellular matrix, and lung extracellular matrix.

7. The composition of claim 4, wherein the synthetic extracellular matrix scaffold component comprises material selected from the group consisting of polyethylene terephthalate fiber (Dacron), polytetrafluoroethylene (PTFE), glutaraldehyde-cross linked pericardium, polylactate (PLA), polyglycol (PGA), hyaluronic acid, polyethylene glycol (PEG), polyethylene glycol diacrylate (PEGDA), polyethelene, a nucleic acid polymer, nitinol, and collagen from non-animal sources.

8. The composition of claim 1, further comprising a cell.

9. The composition of claim 8, wherein the cell is a stem cell.

10. The composition of claim 9, wherein the cell is selected from the group consisting of a human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell.

11. The composition of claim 1, further comprising a peptide, polypeptide or protein.

12. The composition of claim 11, wherein the peptide, polypeptide or protein is conjugated or cross-linked to the extracellular matrix.

13. The composition of claim 11, wherein the peptide, polypeptide or protein is a growth factor.

14. The composition of claim 11, wherein the peptide, polypeptide or protein is selected from the group consisting of a collagen, a proteoglycan, a glycosaminoglycan (GAG) chain, a glycoprotein, a growth factor, a cytokine, a cell-surface associated protein, a cell adhesion molecule (CAM), an angiogenic growth factor, an endothelial ligand, a matrikine, a matrix metalloprotease, a cadherin, an immunoglobin, a fibril collagen, a non-fibrillar collagen, a basement membrane collagen, a multiplexin, a small-leucine rich proteoglycan, decorin, biglycan, a fibromodulin, keratocan, lumican, epiphycan, a heparan sulfate proteoglycan, perlecan, agrin, testican, syndecan, glypican, serglycin, selectin, a lectican, aggrecan, versican, nuerocan, brevican, cytoplasmic domain-44 (CD44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), and vascular epithelial growth factor (VEGF).

15. The composition of claim 1, wherein the damaged or defective intracardiac tissue comprises damage to or a defect in a conduction pathway in the intracardiac tissue.

16. The composition of claim 1, wherein the damaged or defective intracardiac tissue comprises damage to or a defect in tissue of a sino atrial node, an atrio ventricular node, or a conduction pathway in between the two.

17. A composition for reconstruction, replacement or repair of damaged or defective intracardiac tissue comprising at least a portion of an extracellular matrix scaffold component derived from a mammalian or synthetic source and a first additional component selected from the group consisting of a collagen present in myocardial tissue, a collagen type IV protein, a collagen type VIII protein, a collagen type X protein, a multiplexin, a matrix heparin sulfate protoeglycan, a laminin, a nidogen, an agin, and a perlecan, wherein said composition comprises a form selected from the group consisting of a patch, an emulsion, an injectable solution, a gel, a fluid, a paste, a powder, a strand, a strip, a spray, a vapor, a cream, and a coating, and further comprises a second additional component selected from the group of:
a) a cell,
b) a peptide, polypeptide, or protein,
c) a vector having a DNA capable of targeted expression of a selected gene, and
d) a nutrient, a sugar, a fat, a lipid, an amino acid, a nucleic acid, a ribo-nucleic acid, an organic molecule, an inorganic molecule, a small molecule, a drug, or a bioactive molecule.

18. The composition of claim 17, wherein the composition is a patch, and the patch is in a form selected from the group consisting of a sheet, a laminate, a weave, a polymer matrix, a plurality of strands, and one or more strips.

19. The composition of claim 17, wherein the extracellular matrix scaffold component comprises mammalian extracellular matrix.

20. The composition of claim 17, wherein the extracellular matrix scaffold component comprises synthetic extracellular matrix.

21. The composition of claim 17, wherein the extracellular matrix scaffold component comprises a combination of mammalian and synthetic extracellular matrix.

22. The composition of claim 17, wherein said first or second additional component is in a form selected from the group consisting of a gel, an emulsion, an injectable solution, a fluid, a paste, a powder, a strand, a strip, a spray, a vapor, a cream, and a coating.

23. The composition of claim 19, wherein the mammalian extracellular matrix scaffold component comprises extracellular matrix selected from the group consisting of small intestine submucosa (SIS), urinary bladder submucosa (UBS), urinary basement membrane (UBM), liver basement membrane (LBM), stomach submucosa (SS), collagen from animal sources, collagen from plant sources, synthesized extracellular matrix in cultures from cells (Matrigel™), dermal extracellular matrix, subcutaneous extracellular matrix, large intestine extracellular matrix, placental extracellular matrix, ornamentum extracellular matrix, heart extracellular matrix, and lung extracellular matrix.

24. The composition of claim 20, wherein the synthetic extracellular matrix scaffold component comprises a material is selected from the group consisting of polyethylene terephthalate fiber (Dacron), polytetrafluoroethylene (PTFE), glutaraldehyde-cross linked pericardium, polylactate (PLA), polyglycol (PGA), hyaluronic acid, polyethylene glycol (PEG), polyethylene glycol diacrylate (PEGDA), polyethelene, a nucleic acid polymer, nitinol, and collagen from non-animal sources.

25. The composition of claim 17, wherein the first additional component comprises a cell selected from the group consisting of a human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell.

26. The composition of claim 17, wherein the first additional component comprises a peptide, polypeptide, or protein selected from the group consisting of a collagen, a proteoglycan, a glycosaminoglycan (GAG) chain, a glycoprotein, a growth factor, a cytokine, a cell-surface associated protein, a cell adhesion molecule (CAM), an angiogenic growth factor, an endothelial ligand, a matrikine, a matrix metalloprotease, a cadherin, an immunoglobin, a fibril collagen, a non-fibrillar collagen, a basement membrane collagen, a multiplexin, a small-leucine rich proteoglycan, decorin, biglycan, a fibromodulin, keratocan, lumican, epiphycan, a heparan sulfate proteoglycan, perlecan, agrin, testican, syndecan, glypican, serglycin, selectin, a lectican, aggrecan, versican, nuerocan, brevican, cytoplasmic domain-44 (CD-44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), and vascular epithelial growth factor (VEGF).

27. The composition of claim 17, wherein the damaged or defective intracardiac tissue comprises damage to or a defect in a conduction pathway in the intracardiac tissue.

28. The composition of claim 17, wherein the damaged or defective intracardiac tissue comprises damage to or a defect in tissue of a sino atrial node, an atrio ventricular node, or a conduction pathway in between the two.

29. A method of reconstruction, replacement or repair of damaged or defective intracardiac tissue, comprising contacting the damage or defect with the composition of claim 1 or 17.

30. The method of claim 29, wherein contacting comprises surgically opening the patient at a site of damage or defect and placing the composition at the site.

31. The method of claim 30, wherein placing comprises affixing a patch to the damaged or defective intracardiac tissue.

32. The method of claim 31, wherein affixing comprises suturing, stapling, gluing, or tying the patch to intracardiac tissue.

33. The method of claim 29, wherein contacting comprises delivering the composition to a site of damage or defect in the intracardiac tissue using a catheter in a minimally invasive procedure.

34. The method of claim 29, wherein contacting comprises delivering the composition to a site of damage or defect in the intracardiac tissue using a means selected from the group consisting of injecting, injecting with a percutaneous catheter, CO₂ powering emission of fine emulsion, micronizing an injectable solution, and ink jet delivery.

35. A method of making a composition of claim 1 or 17 comprising preparing the composition, and packaging the composition for storage until use.
